(19) United States
(12) Patent Application Publication
(10) Pub. No.: US 2009/0253969 A1
(43) Pub. Date: Oct. 8, 2009

(54) REMOTE PRESSURE-RELEASE SYSTEM

(75) Inventor: Chih-Chen WU, Pingtung County (TW)

Correspondence Address: WPAT, PC, 7225 BEVERLY ST., ANNANDALE, VA, 22003, US

(73) Assignee: Chih-Chen WU, Pingtung County (TW)

(21) Appl. No.: 12/136,834

(22) Filed: Jun. 11, 2008

(30) Foreign Application Priority Data
Apr. 3, 2008 (TW) 097205777

(51) Int. Cl. A61B 5/02; A61H 1/00

(52) U.S. Cl. 600/301; 601/84

(57) ABSTRACT

A remote pressure-release system includes a health-care massage chair, a medical device, and a remote server. The medical device is disposed on the health-care massage chair, and measures physiological conditions of a user sitting on the health-care massage chair, so as to generate a physiological data and output the physiological data to the server. The server, electrically connected to the medical device, is used for analyzes the physiological data from the medical device, and generates a corresponding device parameter to the health-care massage chair. According to the received device parameter, the health-care massage chair controls the massage unit to provide the user the massage services. With this system, the massage services are provided accurately depending upon the users' physiological conditions, and meanwhile, health-care personnel can analyze the users' current health conditions according to the physiological data to determine whether to start relevant subsequent services to improve the serviceability.

This application claims the benefit of Taiwan Patent Application No. 097205777, filed on Apr. 3, 2008, which is hereby incorporated by reference for all purposes as if fully set forth herein.

### BACKGROUND OF THE INVENTION

1. Field of the Invention

The present invention generally relates to a pressure-release system, in particular, to a remote pressure-release system, which includes a massage chair, a medical device, and a remote server, and the medical device is disposed on the massage chair and measures physiological conditions of a user to start relevant massage units.

2. Related Art

Referring to FIG. 1, as for a massage chair 100 in the prior art, one or more massage devices are disposed on an inner side of a chair back, handles, and legs. When a user sits on the massage chair 100, the massage device on the chair back 101 fits the user's back and neck. Likewise, the massage device at the handle 102 fits the user's wrist, and the massage device at the leg 103 fits the user's thigh and calf respectively. All the massage devices vibrate regularly to carry out pressing and releasing actions to the user's neck, back, wrists, and legs repeatedly to a certain extent, thereby providing massaging actions on the user's whole body, which is helpful for tendon relaxation and blood circulation. Furthermore, all the massage devices operate automatically once the massage chair 100 is started, or a control device (not shown) is disposed on the massage chair 100, so as to control operations of all the massage devices.

Furthermore, a media player under the management of a control device is disposed on the massage chair, and a memory module for storing a plurality of music data is built in the massage chair. The user may utilize the control device to control the media player to play any music data and make the massage chair to vibrate according to the melody of the music data, and thus the user becomes relaxed and enjoys the massaging service.

However, the prior art still has unavoidable defects.

Firstly, the massage chair can merely carry out massage actions depending on the mode selected by the user, but the massage mode selected by the user may not be an appropriate operating mode. In addition, when the user feels uncomfortable, an inappropriately-selected massage mode may make the user feel even worse, thereby producing negative effects.

Secondly, although the massage chair is a health-care instrument, it cannot measure the user's physiological conditions. Therefore, the massage chair cannot change the massage mode automatically to cater to the user's physiological conditions to release the user's physical and mental pressures, and cannot prevent the physical hurt caused by the inappropriate usage of the massage chair as well.

### SUMMARY OF THE INVENTION

In view of the above, the present invention provides a remote pressure-release system, which can measure a user's physiological conditions and start a corresponding massage mode, so as to release the user's physical and mental pressures.

In order to solve the above problem, a technical solution provided by the present invention is directed to a remote pressure-release system, which is formed by a health-care massage chair and a server.

The health-care massage chair is disposed with at least one medical device and at least one massage unit. The medical device is electrically connected to the server and measures the physiological conditions of the user sitting on the health-care massage chair, so as to generate a physiological data and output the physiological data to the server.

The server analyzes the physiological data received from the medical device, generates a device parameter corresponding to the physiological data, and outputs the device parameter to the massage unit. Then, the massage unit is actuated according to the device parameter.

The server of the present invention further stores a plurality of mood data. The server analyzes a mood data matched with the physiological data, generates a device parameter corresponding to the mood data, and outputs the device parameter to the massage unit.

The server of the present invention further stores a plurality of media data and the health-care massage chair is further disposed with a media player. The server analyzes a mood data matched with the physiological data and outputs the corresponding media data to the media player for being played.

The server of the present invention further analyzes the physiological data and starts at least one subsequent service corresponding to the physiological data, which includes a health alert, a health-care visit, a clinic reservation, a medication tracing, a residence allocation of drugs, or a diet formulation.

The system of the present invention also has a function of accumulating experience automatically, in which the most comfortable massaging rhythm can be figured out through analyzing the records of customer services, and the optimal pressure-releasing solution can be provided under the condition of the same physiological information through utilizing the accumulated experience values.

The present invention has efficacies that cannot be achieved by the prior art.

Firstly, after the health-care massage chair is started, the medical device measures the physiological data, and then, the server generates the device parameter corresponding to the physiological data according to the received physiological data, thereby controlling the massage unit according to the device parameter. Hence, the user's physical and mental pressures can be released accurately and effectively, and the worthless physical hurt caused by inappropriate usage can be avoided.

Secondly, the user's current mood can be reflected by measuring the physiological conditions of the user, so as to output the corresponding device parameter or relevant media data. If the user is a home-care patient who had better not to change his/her mood dramatically, his/her mood can be effectively smoothed through the system of the present invention, thereby avoiding deterioration of the disease.

Thirdly, when the server analyzes that the user's current physiological conditions are changed dramatically, the disease is deteriorated, or the measured result is excessively abnormal, relevant subsequent services can be started to trace the user's current condition effectively and quickly, and corresponding rescue measures can be taken as well.

### BRIEF DESCRIPTION OF THE DRAWINGS

The present invention will become more fully understood from the detailed description given herein below for illustration only, and thus is not limitative of the present invention, and wherein:

FIG. 1 is a schematic view of a structure in the prior art;

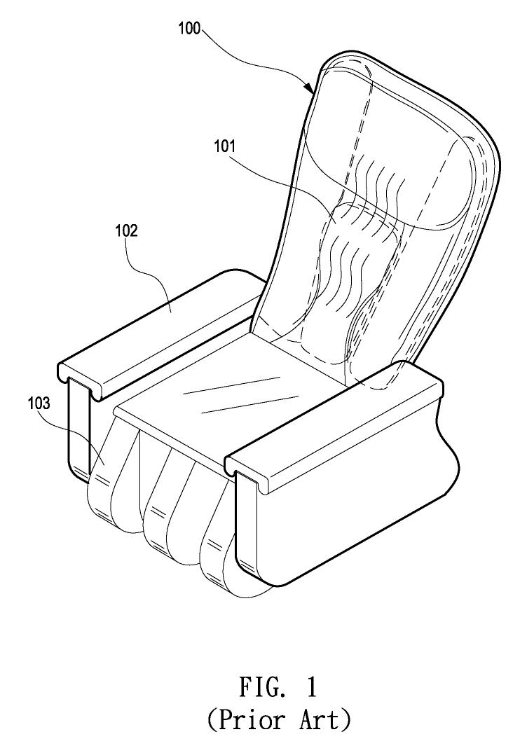

FIG. 2A is an architectural view of a system according to a first embodiment of the present invention;

FIG. 2B is a schematic view of the system according to the first embodiment of the present invention;

FIG. 3A is an architectural view of a system according to a second embodiment of the present invention; and

FIG. 3B is a schematic view of the system according to the second embodiment of the present invention.

### DETAILED DESCRIPTION OF THE INVENTION

In order to further understand the objects, construction features, and functions of the present invention, the specific embodiments and accompanying drawings are illustrated below in detail.

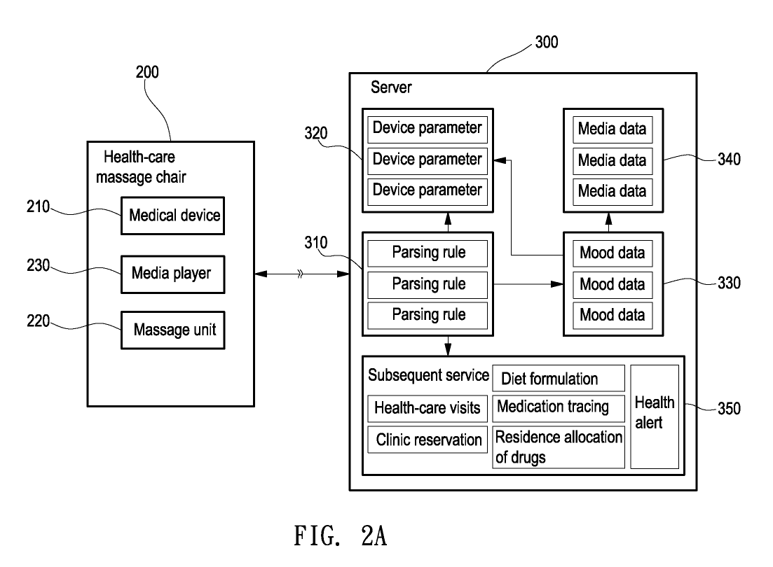

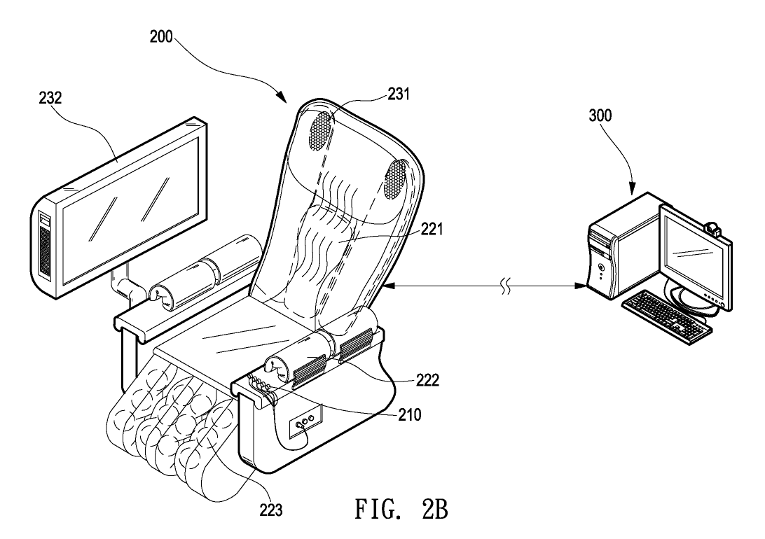

FIGS. 2A and 2B are schematic views of a structure according to a first embodiment of the present invention.

Referring to both FIGS. 2A and 2B, the remote pressure-release system includes a health-care massage chair 200 and a server 300. The health-care massage chair 200 includes at least one medical device 210, at least one massage unit 200, and a media player 230. The server 300 stores one or more device parameters 320, mood data 330, parsing rules 310, and media data 340, and has a function of starting a plurality of subsequent services 350.

The massage unit 200 includes a chair back massage unit 221, a handle massage unit 222, and a leg massage units 223. The chair back massage unit 221 is provided for releasing the pressure on the neck and back of the user, the handle massage unit 222 are provided for releasing the pressure on the palms, wrists, or arms of the user, and the leg massage unit 223 are provided for releasing the pressure on the calves and thighs of the user. All the massage units 220 are connected to the server 300 in a wired or wireless manner. The medical device 210 may be a blood oximeter, a pulse meter, a blood pressure meter, or another different measuring meter, so as to measure the user's physiological conditions and generate a physiological data. The medical device 210 is disposed on each massage unit 220, or additionally disposed on an outer surface of the health-care massage chair 200, and connected to the server 300 in a wired or wireless manner. For example, the blood pressure meter is combined with the handle massage unit 222. Alternatively, the inspecting ends of all the medical devices 210 are configured into a ring shape, and infrared sensors are built in the medical devices 210, so as to carry out physiological measurements from the user's fingers, such as body temperature, pulse, blood oxygen, or blood sugar. Furthermore, the inspecting ends of the medical device 210 may be further configured into a patch for being attached to the skin of any body member of the user, so as to measure the physiological conditions of the user, such as blood sugar, through inspecting the ingredients of the user's sweats.

The media player 230 is mounted on the health-care massage chair 200, and includes a speaker 231 for playing sounds and a display 232 for displaying images.

Once the user starts the health-care massage chair 200, the medical device 210, the player, and the massage unit 220 are all started. The medical device 210 firstly logs on the server 300, and then measures the user's physiological conditions including blood oxygen, blood pressure, and pulse, so as to generate a physiological data and then transmit the physiological data to the server 300. The server 300 retrieves a corresponding parsing rule 310 according to the logon information of the medical device 210, and analyzes the physiological data transmitted from the medical device 210 through utilizing the parsing rule 310. Then, a subsequent service is carried out according to the analysis result, which includes the following three types.

Firstly, the corresponding device parameter 320 is generated according to the parsing result, or one device parameter corresponding to the parsing result is retrieved from the pre-stored device parameters 320. Then, the device parameter is transmitted to the massage unit 220 of the health-care massage chair 200, so that the massage unit 220 is actuated according to the device parameter 320. If it is determined that the blood pulse at the user's legs is relatively weak and the blood pressure is excessively low, it indicates that the user might over use his/her legs. Accordingly, the server 300 outputs and enhances vibrations of the leg massage units 223 and strengthens the massage force for massaging the user's legs, thereby releasing the user's physical pressure effectively.

Secondly, the physiological data is parsed and then the parsing result is compared with the pre-stored mood data 330, so as to determine the user's current mood. Then, the corresponding media data 340 and device parameter 320 are output to the health-care massage chair 200, so that the massage unit 220 is actuated according to the device parameter 320, and the media player 230 plays the media data 340. The media data 340 is formed by at least one of music, pictures, and images, in which the music is played by the speaker 231 of the media player 230, and the pictures and images are displayed by the display 232 of the media player 230. Therefore, the user enjoys the mental-releasing audio-visual services while enjoying the massage services, and thus the user's physical and mental pressures can be released effectively.

Thirdly, the physiological data is parsed and then it is determined whether the user's physiological condition is abnormal or not according to the parsing result, so as to decide whether to start a subsequent service 350 or not. The subsequent service 350 includes health alert, health-care visits, clinic reservation, medication tracing, residence allocation of drugs, or diet formulation. The server 300 starts different subsequent services 350 depending upon the user's different abnormal physiological conditions. Moreover, the physiological data is stored in the server 300, for being used by the health-care personnel or doctors to trace and treat the patient's disease

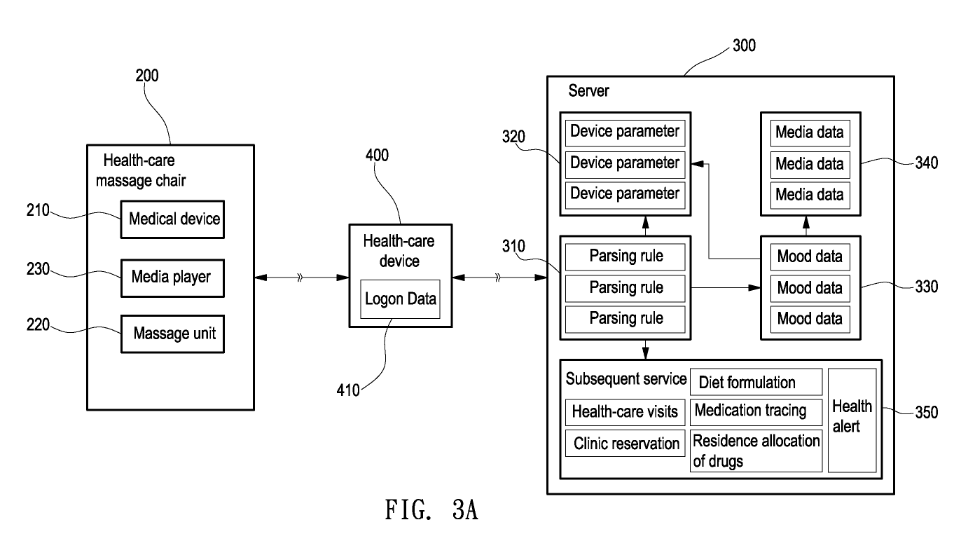

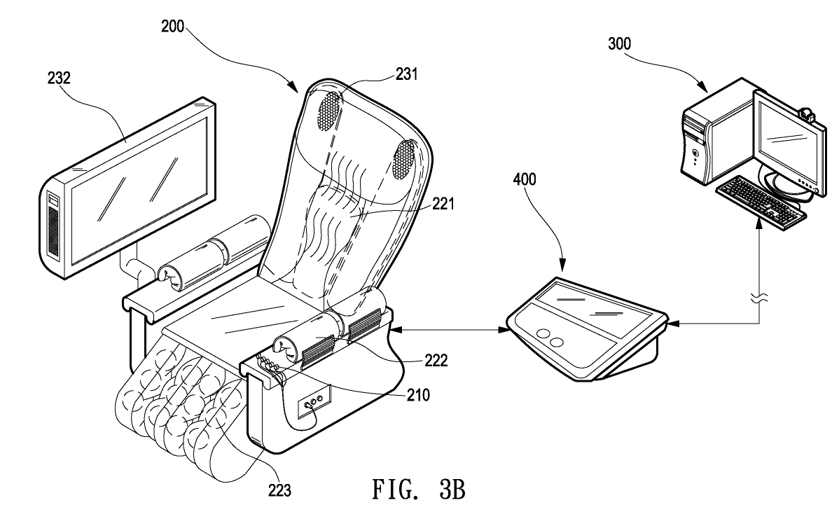

FIGS. 3A and 3B show a second embodiment of the present invention. Referring to both FIGS. 3A and 3B, the difference between the first embodiment and the second embodiment lies in that, the second embodiment further includes a health-care device 400, which is connected to the server 300 in a wired or wireless manner, and the flow for carrying out the remote pressure-release operations is somewhat different from that in the first embodiment.

After the health-care massage chair 200 is started, it is necessary to start the health-care device 400 once again. The health-care device 400 outputs a logon data 410 to log on the server 300, so as to retrieve all the parsing rules 310, mood data 330, and device parameters 320 corresponding to the health-care device 400. The medical device 210 of the health-care massage chair 200 includes a blood oximeter, a pulse meter, or a blood pressure meter, so as to measure the user's physiological conditions and generate a physiological data for being output to the health-care device 400.

The health-care device 400 analyzes the physiological data through using the parsing rules 310, so as to determine the user's current physiological conditions. Meanwhile, the health-care device 400 analyzes the physiological data through using the mood data 330, so as to determine the user's mood. Then, the subsequent service is carried out according to the analysis result, which includes the following three types.

Firstly, according to the parsing result, the device parameter 320 corresponding to the parsing result is output, so that the massage unit 220 is actuated according to the device parameter 320.

Secondly, according to the parsing result of the physiological data and the determination of the user's mood, the corresponding media data 340 and device parameter 320 are output to the health-care massage chair 200. Therefore, the massage unit 220 is actuated according to the device parameter 320, and the media player 230 plays the media data 340.

Thirdly, according to the parsing result of the physiological data, it is determined whether to start the mechanism of the subsequent service 350 or not, such as health alert, health-care visits, clinic reservation, medication tracing, residence allocation of drugs, or diet formulation.

In addition, the health-care massage chair 200 in the first embodiment or the second embodiment can be disposed with a control interface (not shown), which is used for directly controlling the operations of the massage unit 220 and the media player 230. The control interface may be disposed on the display 232 in the form of keys or the display 232 itself is configured into a touch-panel screen, so that the user controls the operations of the health-care massage chair 200 in a touch control manner. Furthermore, the user can directly access the server 300 via the control interface, so as to set, modify, add, and update the user's relevant settings about the health-care massage chair 200 or the health-care device 400 on the server 300.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A remote pressure-release system, comprising:
a health-care massage chair;
at least one medical device, disposed on the health-care massage chair, for measuring physiological conditions of a user sitting on the health-care massage chair, generating a physiological data and outputting the physiological data;
a sever, electrically connected to the medical device, for analyzing the physiological data from the medical device, and generating a device parameter corresponding to the physiological data and outputting the device parameter; and
at least one massage unit, disposed on the health-care massage chair and electrically connected to the server, for being actuated according to the device parameter from the server.

2. The remote pressure-release system according to claim 1, wherein the medical device logs on the server, the server verifies logon information of the medical device and outputs the device parameter corresponding to the medical device according to the physiological data.

3. The remote pressure-release system according to claim 1, wherein the server further stores a plurality of mood data, and the server analyzes the mood data matched with the physiological data, generates the device parameter corresponding to the mood data, and transmits the device parameter to the massage unit.

4. The remote pressure-release system according to claim 3, wherein the server further stores a plurality of media data corresponding to the mood data, the health-care massage chair is further disposed with a media player, and the server analyzes the mood data matched with the physiological data and outputs corresponding media data to the media player for being played.

5. The remote pressure-release system according to claim 4, wherein the media data are selected from a group consisting of music, movies, and pictures.

6. The remote pressure-release system according to claim 1, wherein the server analyzes the physiological data and starts at least one subsequent service corresponding to the physiological data.

7. The remote pressure-release system according to claim 6, wherein the subsequent service comprises a health-care visits, a clinic reservation, a medication tracing, a residence allocation of drugs, or a diet formulation.

8. The remote pressure-release system according to claim 1, wherein the medical device is a blood pressure meter.

9. The remote pressure-release system according to claim 1, wherein the medical device is a blood oximeter.

10. The remote pressure-release system according to claim 1, wherein the medical device is a pulse meter.

11. A remote pressure-release system, comprising:
a health-care massage chair;
at least one medical device, disposed on the health-care massage chair, for measuring physiological conditions of a user sitting on the health-care massage chair and generating a physiological data and outputting the physiological data;
a sever, for storing at least one parsing rule and at least one device parameter;
a health-care device, connected to the server, for retrieving the corresponding parsing rule and the device parameter, analyzing the physiological data from the medical device, and outputting the corresponding device parameter according to an analysis result; and
at least one massage unit, disposed on the health-care massage chair and electrically connected to the health-care device, for being actuated according to the device parameter.

12. The remote pressure-release system according to claim 11, wherein the health-care device logs on the server, and the server verifies and outputs the device parameter and the parsing rule corresponding to the health-care device.

13. The remote pressure-release system according to claim 11, wherein the server further stores a plurality of mood data, and the health-care device retrieves the mood data and analyzes a mood data matched with the physiological data to output the device parameter corresponding to the mood data to the massage unit.

14. The remote pressure-release system according to claim 13, wherein the server further stores a plurality of media data corresponding to the mood data, the health-care massage chair is further disposed with a media player, and the health-care device analyzes the mood data matched with the physiological data, retrieves the corresponding media data from the server, and transmits the media data to the media player for being played.

15. The remote pressure-release system according to claim 14, wherein the media data are selected from a group consisting of music, movies, and pictures.

16. The remote pressure-release system according to claim 11, wherein the server analyzes the physiological data and starts a subsequent service corresponding to the physiological data.

17. The remote pressure-release system according to claim 16, wherein the subsequent service is a health alert, a health-care visits, a clinic reservation, a medication tracing, a residence allocation of drugs, or a diet formulation.

18. The remote pressure-release system according to claim 11, wherein the medical device is a blood pressure meter.

19. The remote pressure-release system according to claim 11, wherein the medical device is a blood oximeter.

20. The remote pressure-release system according to claim 11, wherein the medical device is a pulse meter.
